(19) United States
(12) Patent Application Publication
(10) Pub. No.: US 2026/0306594 A1
(43) Pub. Date: Oct. 8, 2026

(54) LIFTING AGENT WITH THERAPEUTICS

(71) Applicant: Boston Scientific Scimed, Inc., Maple Grove, MN (US)

(72) Inventors: Rosangel Aimee Ramos Espinoza, Boston, MA (US); Carolina Villarreal, Hopedale, MA (US); Katherine Gillig, Hingham, MA (US)

(73) Assignee: Boston Scientific Scimed, Inc., Maple Grove, MN (US)

(21) Appl. No.: 19/639,715

(22) Filed: Apr. 6, 2026

Related U.S. Application Data
(60) Provisional application No. 63/784,660, filed on Apr. 7, 2025.

(51) Int. Cl. A61K 38/48; A61B 90/00; A61K 31/167; A61K 33/38; A61K 38/18; A61K 47/10; A61K 47/38; A61K 47/42; A61K 47/44

(52) U.S. Cl. CPC A61K 38/4886; A61B 90/02; A61K 31/167; A61K 33/38; A61K 38/1858; A61K 47/10; A61K 47/38; A61K 47/42; A61K 47/44

(57) ABSTRACT

In the present disclosure, lifting agents are provided with different therapeutic agents to deliver different therapies. These therapies include chemotherapy, healing promotion, nutritional benefits, and combinations of the same. In some instance, the lifting agent comprises a fluid composition that contains water, at least one biocompatible polymer, and at least one therapeutic agent selected from at least one antitumor agent, at least one wound healing agent, at least one nutritional supplement, and combinations thereof.

### CROSS-REFERENCE TO RELATED APPLICATIONS

This application claims the benefit of U.S. Provisional Patent Application Serial No. 63/784,660 filed on April 7, 2025, the disclosure of which is incorporated herein by reference.

### TECHNICAL FIELD

Embodiments of the present disclosure relate generally to compositions suitable for use as lifting agents, devices and kits comprising such compositions and methods of using such compositions.

### BACKGROUND

Colon cancer is the third most common cancer occurring in the United States. Cells in the colon mucosa have a risk of becoming cancerous, with increasing likelihood as we age. Routine colonoscopies can identify precancerous or cancerous lesions. These lesions can be excised endoscopically. The colon wall can range from 0.2 mm to 3 mm in thickness, and steps are taken to reduce risk of wall perforation during lesion removal. In medical procedures such as endoscopic mucosal resection (EMR) and endoscopic submucosal dissection (ESD), a fluid may be injected into tissue to separate different tissue layers to assist in the removal of lesions and to prevent damage to the underlying colon muscular wall and subsequent perforation. For example, a liquid is commonly injected into a connective tissue layer of the colon (submucosa) to elevate the polyp/lesion away from the rest of the bowel wall. This liquid is commonly referred to as a lifting agent.

### SUMMARY OF THE DISCLOSURE

In the present disclosure, lifting agents are provided with different therapeutic agents to deliver different therapies. These therapies include chemotherapy, healing promotion, nutritional benefits, and combinations of the same.

In some aspects, the lifting agent comprises a fluid composition that contains water, at least one biocompatible polymer, and at least one therapeutic agent selected from at least one antitumor agent, at least one wound healing agent, at least one nutritional supplement, and combinations thereof.

In some embodiments, the at least one therapeutic agent comprises at least one antitumor agent. In some of these embodiments, the at least one antitumor agent is selected from chemotherapy agents, targeted therapy agents, immunotherapy agents, and combinations thereof.

In some embodiments, which can be used in conjunction with the above aspects and embodiments, the at least one therapeutic agent comprises at least one wound healing agent. In some of these embodiments, the at least one wound healing agent is selected from collagenases, lidocaine, hyaluronic acid, silver nanoparticles, platelet-derived growth factors (PDGF), and combinations thereof.

In some embodiments, which can be used in conjunction with the above aspects and embodiments, the at least one therapeutic agent comprises at least one nutritional supplement. In some of these embodiments, at least one nutritional supplement is selected from proteins, fats, and combinations thereof. In some of these embodiments, the at least one nutritional supplement comprises at least one protein and at least one fat and wherein the fat is present in the form of an emulsion.

In some embodiments, which can be used in conjunction with the above aspects and embodiments, the at least one biocompatible polymer is selected from polysaccharides, alkyl celluloses, and alkylene oxide polymers.

In some embodiments, which can be used in conjunction with the above aspects and embodiments, the fluid composition is in the form of a viscous liquid.

In some embodiments, which can be used in conjunction with the above aspects and embodiments, the fluid composition in the form of a shear-thinning gel.

In other aspects, the present disclosure pertains to kits that comprise a container of a fluid composition in accordance with any of the above aspects and embodiments and a delivery device.

In some embodiments, the delivery device comprises a flexible tube and a needle and/or sprayer.

In some embodiments, the fluid composition is provided in a syringe barrel and/or a vial.

In other aspects, procedures are provided that comprise injecting a fluid composition in accordance with any of the above aspects and embodiments through a needle into a target treatment site between an upper mucosal layer and a lower layer such that the upper mucosal layer is separated and elevated from the lower layer, and removing tissue raised by the fluid composition.

In some embodiments, the procedure further comprises applying coating of a fluid composition in accordance with any of the above aspects and embodiments to a site where the tissue has been removed.

The above and other aspects, embodiments, features and benefits of the present disclosure will be readily apparent from the following detailed description.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIG. 1 shows an exemplary medical device in accordance with certain aspects of the present disclosure.

FIGS. 2A-2F illustrate an exemplary tissue resection procedure in accordance with certain aspects of the present disclosure.

### DETAILED DESCRIPTION

Particular aspects of the present disclosure are described in greater detail below.

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass ± 5% of a specified amount or value. The term “exemplary” is used in the sense of “example” rather than “ideal.”

The present disclosure provides lifting agents, which are provided in the form of fluid compositions that comprise water, one or more biocompatible polymers, and one or more therapeutic agents.

Therapeutic agents for use in the fluid compositions of the present disclosure include antitumor agents, wound healing agents, and nutritional supplements.

Antitumor agents are provided in the fluid compositions to kill tumor cells and/or to inhibit growth of tumor cells. The antitumor agent may serve as a prophylactic agent to prevent potential dissemination of cancerous or suspicious cells to other healthy organs or tissues, thereby potentially reducing the incidence of new polyps and tumors, minimizing clinical interventions.

Antitumor agents for use in the present disclosure include chemotherapy agents such as alkylating agents, antimetabolites, platinum compounds, etoposides, topoisomerase inhibitors, nitrosoureas, antimitotic agents, and microtubule inhibitors, among others, targeted therapy agents such as monoclonal antibodies, angiogenesis inhibitors, proteasome inhibitors, and PARP inhibitors, among others, immunotherapy agents such as checkpoint inhibitors, cytokines, monoclonal antibodies, immunomodulatory agents, T-cell agonists, and STING (stimulator of interferon genes) agonists, among others, as well as combinations two or more of the foregoing antitumor agents.

Antitumor agents may be introduced into the fluid compositions during manufacture, or they may be introduced by a medical professional at the time of the clinical procedure, giving the medical professional the ability to adjust the concentration of the antitumor drug as applicable.

Wound healing agents are provided in the fluid compositions of the present disclosure to help the submucosa and/or mucosa layer heal after a lesion is resected, which may speed up patient recovery times and/or prevent delayed bleeding. In addition, because the wound healing agents and the other therapeutic agents described herein are beneficial to the patent, health care providers do not have to spend additional time removing excess material. In fact, it is desirable that at least a portion of the composition remains in place after the resection.

Wound healing agents for use in the fluid compositions of the present disclosure may range include collagenases, lidocaine, hyaluronic acid, silver nanoparticles, platelet-derived growth factors (PDGF), and combinations of two or more of same, among others. Wound healing agents may be introduced into the fluid compositions during manufacture, or they may be introduced by a medical professional at the time of the clinical procedure, giving the medical professional the ability to adjust the concentration of the wound healing agents as applicable.

Nutritional supplements are provided in the fluid compositions of the present disclosure, for example, to buffer the period when patients are on a liquid diet after the procedure. A liquid diet may not provide sufficient nutrition for certain patients compared to their typical diet, so supplements are provided to compensate for this deficiency and reduce the risk of extended patient recovery times due to improper nutrition. The nutritional supplements are selected to migrate from the lifting agent into the intestine wall.

Nutritional supplements include proteins and/or and fats which could enhance postoperative recovery of patients with gastric cancer, as issues associated with deficiencies in these macronutrients are often seen in these patients. Full-liquid perioperative diets often fall short in protein content. Protein intake influences patient serum albumin levels, and low serum albumin is a leading postoperative complication for gastric cancer patients (see Range TL, Samra NS. Full Liquid Diet. [Updated 2023 Mar 6]. In: StatPearls [Internet]. Treasure Island (FL): StatPearls Publishing; 2025 Jan-. Available from: https://www.ncbi.nlm.nih.gov/books/NBK554389/). Another leading postoperative complication for gastric cancer patients is weight loss (Id.). Fats are high-calorie macronutrient that have the potential to enhance patient weight gain. Fats provide about 9 kcal per gram, while other macronutrients such as carbohydrates only provide about 4kcal per gram.

Protein is generally water-soluble and fat is generally water insoluble, so the approaches to providing them in the fluid composition may differ as described below. Although nutritional supplements are generally introduced into the fluid compositions during manufacture, they may also be introduced by a medical professional at the time of the clinical procedure, giving the medical professional the ability to adjust the concentration and type(s) of nutritional supplements as applicable.

Examples of nutritional supplements include proteins such as whey protein, whey isolates, casein, soy protein, egg white, hemp proteins, pea protein, and combinations of the same, among others, and fats include fats from sources such as milk, nuts, seeds, and combinations of the same, among others.

Water may be supplied in the fluid compositions of the present disclosure in a variety of forms including water for injection, normal saline, or phosphate buffered saline, among others.

Because the fluid compositions of the present disclosure are water-based, hydrophilic agents can be dissolved or otherwise dispersed in the aqueous phase that is present. Hydrophobic agents, on the other hand may be insoluble in the aqueous phase and may require emulsification such that they are suspended and dispersed in the aqueous phase.

Biocompatible polymers are included in the fluid compositions of the present disclosure to promote the formation of viscous liquids or gels, among other potential benefits. While viscous liquids and gels are both forms of matter that can flow, viscous liquids flow and conform to the shape of their container whereas gels are semi-solid, with water dispersed within a three-dimensional network, providing resistance to flow. Many polymer-containing viscous liquids exhibit shear-thinning behavior, which means that their viscosity decreases as applied shear (e.g., shear stress and/or shear rate) increases. Similarly, various gels are shear-thinning gels, which become more fluid when subjected to shear (e.g., when injected from a syringe) and then regain their original gel state when the stress is removed. Shear-thinning gels are desirable as injectable materials because they can flow under shear stress during injection and then recover their original gel state when the shear stress is removed. Shear-thinning fluids are also known as pseudoplastic fluids. Both viscous liquids and gels provide a desired lift and persistence for EMR/ESD.

Biocompatible polymers for use in the present disclosure may be natural or synthetic in origin, and may be anionic, cationic, or neutral in charge. Non-limiting examples of biocompatible polymers include polysaccharides, including alkyl celluloses such as methylcellulose, hydroxypropyl methylcellulose, carboxymethylcellulose, anionic polysaccharides such as hyaluronic acid, alginic acid, xanthan gum, gellan gum and gum arabic, cationic polysaccharides such as chitosan, pectin, agar, dextran, polyalkylene oxides including poly(ethylene oxide) (also referred to as polyethylene glycol (PEG)), poly(propylene oxide) (also referred to as polypropylene glycol (PPG)), copolymers of ethylene oxide and propylene oxide (e.g., poloxamers), poly(hydroxyethyl methacrylate), poly-2-dimethylaminoethyl methacrylate, poly-L-lactic acid, poly(lactic-co-glycolic acid), polycaprolactone, polyoxazolines, poly(N-isopropylacrylamide) and copolymers thereof, polyvinyl alcohol, poly(amino acids), including proteins such as collagen, gelatin, heparin, other peptide-based materials, or a combination of two or more of any of the foregoing.

In some embodiments, the fluid compositions of the present disclosure may comprise one or more biocompatible salts. Non-limiting examples of salts suitable for the compositions herein include salts comprising sodium, calcium, magnesium, and/or potassium cations. The salts may include, for example, chloride salts, phosphate salts, and/or sulfate salts, such as, e.g., sodium chloride (NaCl), potassium chloride (KCl), calcium chloride (CaCl₂), sodium dihydrogen phosphate (NaH₂PO₄), potassium hydrogen phosphate (K₂HPO₄), magnesium sulfate (MgSO₄), sodium gluconate (C₆H₁₁NaO₇), sodium acetate trihydrate (C₂H₉NaO₅·3H₂O), and magnesium chloride (MgCl₂). For example, the fluid compositions may comprise an anionic polysaccharide, and the salt(s) may provide a source of monovalent or divalent cations compatible with the polysaccharide to form a viscous liquid or gel.

In some embodiments, the fluid compositions may be isotonic, for example, having an osmolality ranging from about 240 mOsmol/kg to about 340 mOsmol/kg (e.g., 290 mOsmol/kg ± 50 mOsmol/kg), such as from about 250 mOsmol/kg to about 320 mOsmol/kg, or from about 280 mOsmol/kg to about 300 mOsmol/kg. For example, the composition may have concentration of monovalent and/or divalent cations such that the composition is isotonic with tissue fluids and/or blood. Physiologically-compatible solutions comprising other suitable solutes may also be used to provide for isotonicity.

In some embodiments, the fluid compositions of the present disclosure may have pH suitable for injection into a patient (e.g., a pH ranging from 4 to 9). The pH of the solution may be adjusted using a suitable base such as sodium hydroxide to increase pH and/or a suitable acid such as hydrochloric acid, or may adjusted by other means or with other substances providing for a biocompatible composition.

The fluid compositions of the present disclosure may comprise one or more other biocompatible compounds or agents. For example, the compositions may comprise a biocompatible dye or coloring agent, such as brilliant blue (e.g., Brilliant Blue FCF, also known as FD&C Blue 1) indigo carmine (also known as FD&C Blue 2), indigo carmine lake, FD&C Blue 1 lake, and methylene blue (also known as methylthioninium chloride) or a fluorescent dye such as fluorescein, indocyanine green, or fluorescent proteins (e.g. green, blue, cyan fluorescent proteins). For example, the compositions may comprise a dye or colorant to allow for identification of the submucosal tissue plane upon injection into tissue, e.g., to determine the amount of tissue to be removed and/or assess the risk of perforation. Any other suitable types of biocompatible agents may be used, e.g., to adjust the pH and/or tonicity of the composition as appropriate for injection into tissue. In some embodiments, the composition may comprise one or more suspension agents, stabilizers and/or preservatives. In some embodiments, the compositions may comprise a hemostatic additive such as epinephrine to limit superficial bleeding.

Reservoirs suitable for the fluid compositions described herein may include, for example, syringes (e.g., a syringe barrel compatible with a manual or automatic injection system), flexible pouches such as a plastic bag, vials including septum vials, and other fluid containers configured for use with a suitable injection needle or sprayer. Exemplary materials suitable for the reservoir include, but are not limited to, cyclic olefin copolymer, cyclic olefin polymer, polypropylene, polycarbonate, polyvinyl chloride, and glass.

The reservoir may be directly coupled to a needle or sprayer, e.g., via a luer adapter or other suitable connection, or may be indirectly coupled to a needle or sprayer via a flexible tube, such as a catheter. Non-limiting examples of needles coupled a reservoir via a flexible tube include Interject™ sclerotherapy needles by Boston Scientific. The needle may be a hypodermic needle, and may range from a size of 7 gauge (4.57mm outer diameter (OD), 3.81mm inner diameter (ID)) to 33 gauge (0.18mm OD, 0.08mm ID), e.g., a size of 16 gauge (1.65mm OD, 1.19mm ID), 21 gauge (0.82mm OD, 0.51mm ID), 22 gauge (0.72mm OD, 0.41mm ID), 23 gauge (0.64mm OD, 0.33 ID), or 24 gauge (0.57mm OD, 0.31mm ID). Exemplary materials for the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a luer adapter) for engagement with a syringe or other reservoir. In some embodiments, the needle may include an elongated tube (e.g., a catheter) between the needle tip and the proximal fitting/adapter.

The composition may be sterilized. For example, the composition may be autoclaved while inside the reservoir by heating the composition at or to a temperature of about 121°C. Additionally or alternatively, the composition may be sterilized via gamma irradiation or by electron beam after its introduction into the reservoir.

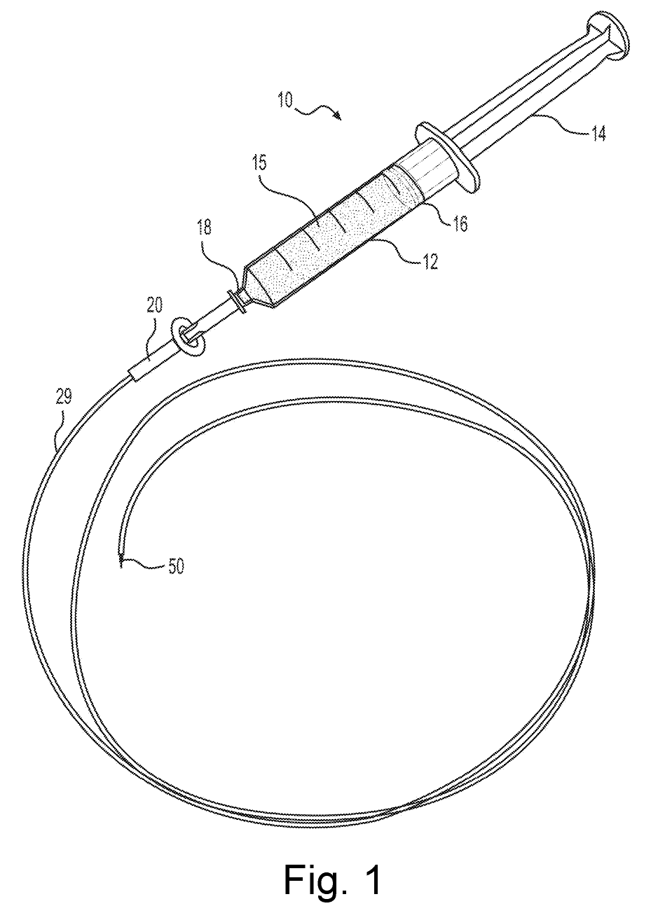

FIG. 1 illustrates an exemplary syringe 10 providing a reservoir for a fluid composition as described herein. The syringe 10 may comprise a barrel 12, a plunger 14, and one or more stoppers 16. The barrel 12 may include a luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing the fluid composition 15 for injection through the needle 50.

Reservoirs other than those illustrated in FIG. 1 may be used in according with the present disclosure. For example, the composition may be housed in a reservoir coupled to a fluid channel and/or needle that forms part of an electrocautery device or system. Thus, a physician may inject the composition through the fluid channel while simultaneously or subsequently operating other portions of the device or system, such as an electrocautery knife or snare.

According to some aspects of the present disclosure, the size of the needle may be chosen based on the viscosity and/or components of the composition, or vice versa. Further, the dimensions of the catheter tubing (inner diameter, outer diameter, and/or length), if any, may affect the types and amount of force applied to the composition during injection. These parameters may be taken into consideration according to the properties of the composition and the needs of the patient. According to some aspects of the present disclosure, the size of the needle may be 23 gauge, 24 gauge, or 25 gauge. In some cases, a larger size of 20 gauge, 21 gauge, or 22 gauge may be used to inject the compositions herein.

The fluid compositions described herein may be used in various medical procedures, including tissue resection procedures of the GI system, the respiratory system, and/or the genitourinary system. The tissue resected in such medical procedures may comprise diseased or injured tissue, non-diseased tissue, or a combination thereof. Exemplary tissue resection procedures include endoscopic mucosal resection (EMR) and endoscopic submucosal dissection (ESD). In these procedures, an endoscope is typically inserted into the patient’s esophagus or anus and advanced through the GI system to reach the target site in the esophagus, stomach, or intestines. EMR is typically used for removal of tissue smaller than 2 cm in diameter, e.g., to biopsy tissue or to remove injured or diseased tissue (e.g., a cancerous lesion), while ESD is typically used for removal of larger lesions.

In some embodiments, a fluid composition as described herein may be injected between two layers of tissue, e.g., injected into submucosal tissue between an upper mucosal layer and lower muscularis propria layer at a target treatment site. The composition may be injected within the submucosal space (submucosal layer) under a portion of tissue, whereupon the injected composition may cause the mucosal tissue to separate from the muscularis propria layer, elevating the mucosal tissue layer. A suitable cutting device such as a knife, snare, scissors, or forceps, e.g., an electrocautery cutting device such as an electrocautery knife, snare, scissors, or forceps, may then be used to remove the portion of tissue. For removal of larger portions of tissue (e.g., via ESD), the composition may be injected under the portion of tissue, wherein the fluid composition elevates the upper layer of tissue from the lower layer. The cutting device then may be used to make an incision around the portion of tissue and remove it. The composition may be injected in the submucosal layer to assist in removing additional portions of tissue.

In some embodiments, the composition may maintain separation of the tissue layers throughout the entire resection procedure. A portion of the composition may be removed via the resection process. Following tissue resection, remaining portions of the composition remain in contact with the tissue, thereby allowing therapeutic agents within the composition to be released onto and/or into the adjacent tissue.

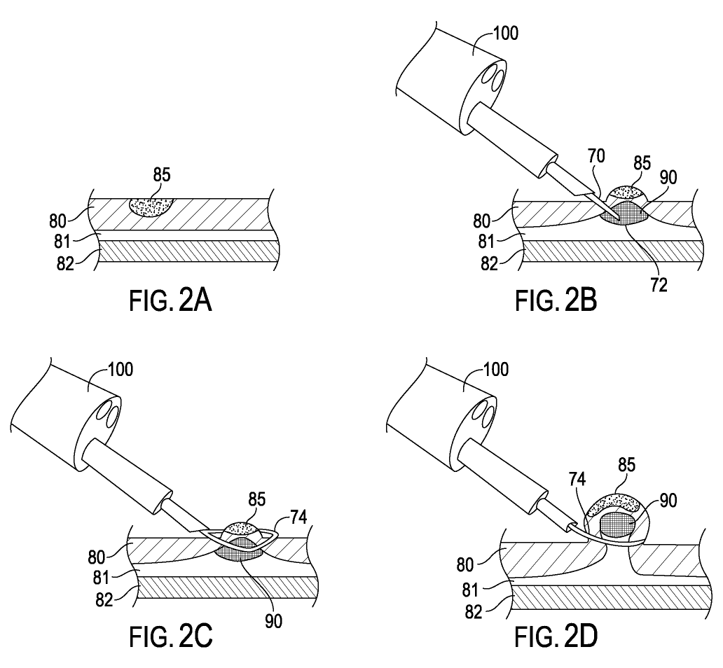

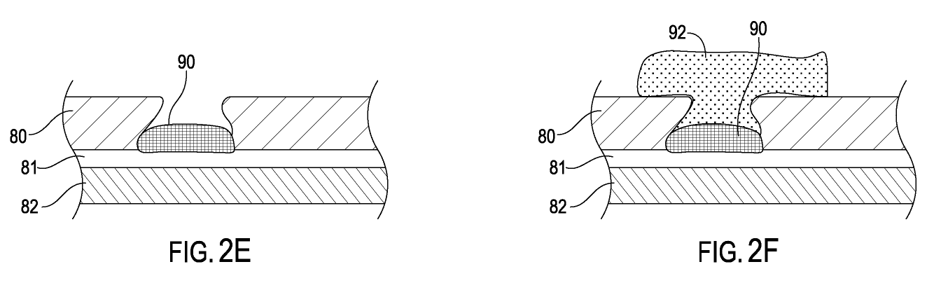

FIGS. 2A-2E illustrate an exemplary resection procedure according to embodiments of the present disclosure. Patients include mammalian patients, particularly human patients. The procedure may be, for example, EMR or ESD as discussed above, or any other suitable medical procedure for resecting tissue. FIG. 2A shows a cross-sectional view of two portions of tissue or tissue layers 80, 82, which may be separated by a middle layer 81 of tissue (such as, e.g., upper mucosal and lower muscularis propria layers separated by a middle submucosal tissue layer). One or both of the portions of tissue 80, 82 may include a section of tissue 85 targeted for removal. For example, the section of tissue 85 may comprise injured or diseased tissue or may comprise tissue targeted for biopsy and subsequent analysis. In the example of FIG. 2A, the section of tissue 85 is located toward the tissue surface, however, the devices and compositions disclosed herein may be used to remove tissue from inner tissue layers.

As shown in FIG. 2B, an endoscope 100 defining one or more lumens (e.g., three lumens as shown) may be used to deliver a needle 70 to the treatment site. The needle 70 may have a hollow lumen and a sharp, beveled tip 72 for piercing the tissue surface such that the needle tip 72 is within the middle layer 81 between the upper and lower portions of tissue 80, 82. The needle lumen may be in communication with a fluid reservoir, such as a syringe or other reservoir containing a fluid composition as described herein. The syringe may be used to inject the composition 90 into the middle layer 81 between the portions of tissue 80, 82 to form a cushion or bleb of material, as shown in FIG. 2B. Once the composition 90 is injected, the volume of the composition 90 may cause the upper and lower portions of tissue 80, 82 to separate, such that the section of tissue 85 may be elevated from underlying tissue. An electrocautery snare 74 or other cutting device 74 (such as, e.g., an electrocautery knife, scissors, or forceps, among other suitable cutting devices) may be used to cut and remove the section of tissue 85, as shown in FIGS. 2C and 2D. Once the section of tissue 85 is removed, as shown in FIG. 2E, a portion of the composition 90 remains in contact with one or more of the tissue layers 80, 81, 82.

In some embodiments, as shown in FIG. 2F, a fluid composition as described herein may be extruded, sprayed, or otherwise applied to the site of the resection to form a coating over the resection site. As shown in FIG. 2F the post-resection composition 92 (e.g., coating) remains in contact with tissue layer 80 and the pre-resection composition 90. The post-resection composition 92 and pre-resection composition 90 may contain the same or different therapeutic agents.

Although the post-resection composition 92 is only in contact with tissue layer 80 in FIG. 2F, in other embodiments, the post-resection composition 92 may be contact with one or more of the tissue layers 80, 81, 82.

As previously indicated, in some embodiments, the pre-resection composition 90 and/or the post-resection composition 92 that remain after the procedure contain one or more include antitumor agents. Therefore, once the tissue is resected, the submucosa and surrounding tissue is left covered with at least one antitumor-agent-containing composition, and the antitumor agents in the remaining compositions are present to treat the tissue to either kill cancerous cells or inhibit the tumor from growing. Additionally, such an approach could also be an alternative delivery route for anticancer agents, as they are typically taken by mouth or injected into the bloodstream via periodic infusions in outpatient centers. In this way, the anticancer agents may serve as a neoadjuvant or adjuvant therapy, that will complement the traditional standard of care and could augment patient outcomes while maximizing therapy efficacy due to its proximity to the affected area.

As also previously indicated, in some embodiments, the pre-resection composition 90 and/or the post-resection composition 92 may contain one or more wound healing agents to help the submucosa and/or mucosa layer heal after resection and/or to speed up patient recovery times and/or prevent delayed bleeding. The pre-resection composition 90 and/or the post-resection composition 92 may also contain one or more nutritional supplements to provide nutritional therapy.

In various embodiments, the pre-resection composition 90 and/or the post-resection composition 92 may contain any of the following combinations of therapeutic agents: (a) one or more antitumor agents and one or more wound healing agents, (b) one or more antitumor agents and one or more nutritional supplements, (c) one or more wound healing agents and one or more nutritional supplements, and (d) one or more antitumor agents, one or more wound healing agents, and one or more nutritional supplements.

Other aspects and embodiments of the present disclosure will be apparent to those skilled in the art from consideration of the specification and practice of the embodiments disclosed herein. While certain features of the present disclosure are discussed within the context of exemplary tissue resection procedures, the compositions, systems, and methods may be used for other medical procedures according to the general principles disclosed.

1. A lifting agent comprising a fluid composition that contains water, at least one biocompatible polymer, and at least one therapeutic agent selected from at least one antitumor agent, at least one wound healing agent, at least one nutritional supplement, and combinations thereof.

2. The lifting agent of claim 1, wherein the at least one therapeutic agent comprises at least one antitumor agent.

3. The lifting agent of claim 2, wherein the at least one antitumor agent is selected from chemotherapy agents, targeted therapy agents, immunotherapy agents, and combinations thereof.

4. The lifting agent of claim 1, wherein the at least one therapeutic agent comprises at least one wound healing agent.

5. The lifting agent of claim 4, wherein the at least one wound healing agent is selected from collagenases, lidocaine, hyaluronic acid, silver nanoparticles, platelet-derived growth factors (PDGF), and combinations thereof.

6. The lifting agent of claim 1, wherein the at least one therapeutic agent comprises at least one nutritional supplement.

7. The lifting agent of claim 6, wherein the at least one nutritional supplement is selected from proteins, fats, and combinations thereof.

8. The lifting agent of claim 6, wherein the at least one nutritional supplement comprises at least one protein and at least one fat and wherein the fat is present in the form of an emulsion.

9. The lifting agent of claim 1, wherein the at least one biocompatible polymer is selected from polysaccharides, alkyl celluloses, and alkylene oxide polymers.

10. The lifting agent of claim 1, wherein the fluid composition is in the form of a viscous liquid.

11. The lifting agent of claim 1, wherein the fluid composition in the form of a shear-thinning gel.

12. A kit comprising a delivery device and a fluid composition that contains water, at least one biocompatible polymer, and at least one therapeutic agent selected from at least one antitumor agent, at least one wound healing agent, at least one nutritional supplement, and combinations thereof.

13. The kit of claim 12, wherein the delivery device comprises a flexible tube and a needle and/or sprayer.

14. The kit of claim 12, wherein fluid composition is provided in a syringe barrel and/or a vial.

15. The kit of claim 12, wherein the at least one therapeutic agent comprises at least one antitumor agent.

16. The kit of claim 15, wherein the at least one antitumor agent is selected from chemotherapy agents, targeted therapy agents, immunotherapy agents, and combinations thereof.

17. The kit of claim 12, wherein the at least one therapeutic agent comprises at least one wound healing agent.

18. The kit of claim 12, wherein the at least one therapeutic agent comprises at least one nutritional supplement.

19. A procedure comprising injecting a fluid composition through a needle into a target treatment site between an upper mucosal layer and a lower layer such that the upper mucosal layer is separated and elevated from the lower layer, and removing tissue raised by the fluid composition, the fluid composition comprising water, at least one biocompatible polymer, and at least one therapeutic agent selected from at least one antitumor agent, at least one wound healing agent, at least one nutritional supplement, and combinations thereof.

20. The procedure of claim 19, further comprising applying coating of the fluid composition to a site where the tissue has been removed.
